(19) United States
(12) Patent Application Publication
(10) Pub. No.: US 2008/0226699 A1
(43) Pub. Date: Sep. 18, 2008

(54) Method and System for Rapid Transdermal Administration

(75) Inventors: Barrie Charles Finnin, Victoria (AU); Timothy Matthias Morgan, Victoria (AU); Nina Frances Wilkins, Victoria (AU); Nora Yat Knork Chew, Victoria (AU); Igor Gonda, Victoria (AU)

Correspondence Address: FOLEY AND LARDNER LLP;SUITE 500, 3000 K STREET NW, WASHINGTON, DC, 20007, US

(73) Assignee: Acrux DDS Pty Ltd

(21) Appl. No.: 10/580,845

(22) PCT Filed: Nov. 26, 2004
(86) PCT No.: PCT/AU2004/001666

§ 371 Date: Apr. 9, 2008

(30) Foreign Application Priority Data
Nov. 28, 2003 (AU) 2003906631

(51) Int. Cl. A61M 37/00; A61K 31/445

(52) U.S. Cl. 424/449; 514/329

(57) ABSTRACT

Invention relates to a method for transdermal delivery of a topically applied physiologically active agent comprising: providing a micro-projection apparatus comprising an array of microprojections (3) extending from a substrate; applying the array of micro-projections to an area of skin to form an array of microscopic holes therein; and contacting the area of skin with a transdermal composition comprising a physiologically active agent and at least one penetration enhancer wherein the formation of the microscopic holes and penetration enhancer facilitate transdermal delivery of the physiologically active agent.

### FIELD

The present invention relates to a method and system for the treatment of an animal, including humans, requiring a rapid therapeutic effect following transdermal or topical drug delivery, whereby the method the invention provides a method for enhancing the uptake of a drug in to the systemic circulation.

### BACKGROUND

There is a constant need for methods for the safe and effective administration of physiologically active agents. For many medications it is important that the administration regime is as simple and non-invasive as possible in order to maintain a high level of compliance by a patient. Oral administration is one administration regime that is commonly used because it is a relatively simple regime to follow. However, the oral administration route is also complicated because of complications associated with gastrointestinal irritation and with drug metabolism in the liver and its consequent reliance on the use of higher oral doses than would be otherwise required with transdermal or other forms of direct systemic delivery.

For many symptoms of a disease or condition, a rapid drug effect is desirable. Drug delivery via injection is traditionally the quickest route of administration into the systemic circulation, however, the duration of action is often short lived and the mode of delivery invasive and painful. Administration of physiologically active agents through the skin (‘transdermal drug delivery’) has received increased attention because it not only provides a relatively simple dosage regime but it traditionally provides a relatively slow and controlled route for release of a physiologically active agent into the systemic circulation. However, transdermal drug delivery is complicated by the fact that the skin behaves as a natural barrier and therefore transport of agents through the skin is a complex mechanism.

Rapid onset following transdermal or topical drug delivery would offer several inherent clinical and patient advantages over the traditional injection in that it is non-invasive, will increase patient compliance with no pain, will retain a controlled and sustained drug delivery, and can be self administered.

Structurally, the skin consists of two principle parts, a relatively thin outermost layer (the ‘epidermis’) and a thicker inner region (the ‘dermis’). The outermost layer of the epidermis (the ‘stratum corneum’) consists of flattened dead cells which are filled with keratin. The region between the flattened dead cells of the stratum corneum is filled with lipids which form lamellar phases that are responsible for the natural barrier properties of the skin. Epidermal thickness varies between 60 and 800 μm depending on anatomical site, cell size and the number of cell layers, with the stratum corneum being between 0.5 μm and 20 μm thick. (Mackenzie J, 1969, Nature 222: 881-882. Barry B W, 1983, in: Percutaneous absorption, New York, Marcel and Dekker: ch. 1).

For effective transdermal delivery of a therapeutic agent that is applied to the surface of the skin (‘topical application’), the agent must be partitioned firstly from the vehicle into the stratum corneum, it must typically then be diffused within the stratum corneum before being partitioned from the stratum corneum to the viable epidermis and dermis and then into the bloodstream.

To overcome some of the problems with transdermal delivery that are associated with transport across the dermal layers (‘percutaneous absorption’), physiologically active agents are commonly formulated with incorporation of one or more drug penetration enhancers which are often lipophilic chemicals that readily partition into the stratum corneum whereupon they exert their effects on improving the transport of drugs across the skin barrier. For example, U.S. Pat. No. 6,299,900 to Reed et al. describes safe sunscreen ester dermal penetration enhancers such as octyl salicylate (octisalate) for the enhanced transdermal delivery of physiologically active agents.

Alternatively, methods that allow a drug to penetrate into the viable epidermis by breaching the stratum corneum have been employed. The microneedle devices disclosed in the prior art often include a reservoir which provides a supply of the drug to be administered transdermally. The reservoir is in many cases, located within a patch which is provided with microneedles and the drug is fed into the microneedles by a lumen within the needle itself or from the underside of the patch. For example U.S. Pat. No. 6,503,231 (Prausnitz et al) discloses a microneedle device which contains hollow or porous microneedles and which allow a drug to penetrate into the viable epidermis by breaching the stratum corneum.

In order to be effective, the microneedles must penetrate the rate limiting barrier, the stratum corneum, whilst minimising the depth of penetration such that the lower layers of the viable epidermis is not breached, thus pain and bleeding is avoided. One of the objectives of many of the prior art documents cited is to obtain consistent and predictable depth of penetration. WO98/28037 generally uses an array of microblades for piercing and anchoring the skin for increased transdermal flux of an agent. WO03/053258 further describes a piercing micro-projection which has a depth penetration control such as a shoulder. However, less attention has been given to the effect of pressure loading on the depth of penetration, and to the effect of the composition formulation on penetration of the abraded surface, thus inconsistent and unpredictable drug delivery is likely to occur.

There is a need for simple, effective application of a transdermal or topical composition into the skin and/or systemic circulation where rapid onset is desirable.

No admission is made that any reference, including any patent or patent document, cited in this specification constitutes prior art. In particular, it will be understood that, unless otherwise stated, reference to any document herein does not constitute an admission that any of these documents forms part of the common general knowledge in the art in Australia or in any other country. The discussion of the references states what their authors assert, and the applicant reserves the right to challenge the accuracy and pertinency of any of the documents cited herein.

### SUMMARY

The present invention arises from the inventor's studies of transdermal and topical formulations which contain penetration enhancers that enhance the percutaneous absorption of a physiologically active agent. The inventor's studies have shown that the extent of release of physiologically active agent may be further enhanced to provide for rapid transdermal drug delivery.

The present invention provides a method for enhancing the percutaneous absorption of a physiologically active agent thereby enabling rapid elevated drug serum concentrations to be provided within the bloodstream of an animal.

Accordingly, in a first aspect the present invention provides a method of treatment wherein rapid systemic drug delivery is achieved the method comprising applying to an area of skin of an animal a microneedle device.

In a second aspect the invention provides the use of a microneedle device in preparation of a transdermal delivery system for rapid systemic drug delivery by application of a drug delivery system to the skin of an animal.

The method of the invention preferably includes the step of applying a microneedle device under a predefined and reproducible pressure load to the transdermal application site.

In a third aspect the invention provides a method for transdermal delivery to an animal of a topically applied physiologically active agent, the method comprising:
providing a microprojection apparatus comprising an array of microprojections extending from a substrate;
applying the array of microsprojections to an area of skin of the animal to form an array of microscopic holes therein; and
contacting the area of skin with a transdermal composition comprising a physiologically active agent and at least one penetration enhancer
wherein the formation of the microscopic holes and the penetration enhancer facilitate transdermal delivery of the physiologically active agent.

In a fourth aspect the invention provides a method for transdermal drug delivery system which comprising
a micro-projection apparatus comprising:
   a cuff for encircling a body member;
   an array of micro-projections extending inwardly from the cuff;
   a means for applying a predetermined constricting force to the cuff when placed about the body member thereby causing the array of micro-projections to penetrate the stratum corneum; and
a transdermal composition comprising:
   a physiologically active agent; and
   at least one dermal penetration enhancer; and
wherein the micro-projection apparatus facilitates transdermal penetration of the transdermal composition.

In a fifth aspect the present invention provides a transdermal drug delivery system comprising a micro-projection apparatus comprising:
a cuff for encircling a body member;
an array of micro-projections extending inwardly from the cuff;
a means for applying a predetermined constricting force to the cuff when placed about the body member thereby causing the array of micro-projections to penetrate the stratum corneum; and
a transdermal composition comprising:
a physiologically active agent; and
at least one dermal penetration enhancer; and
wherein the micro-projection apparatus facilitates transdermal penetration of the transdermal composition.

The micro-projection apparatus may be applied to the area of skin through which transdermal penetration is to occur before during or after application of the transdermal composition.

### DETAILED DESCRIPTION

The use of a microneedle apparatus together with a transdermal composition containing a penetration enhancer has been found to provide further rapid uptake of the drug when compared to compositions without a penetration enhancer. This is particularly useful for treatments such as pain relief wherein an immediate physiological effect is desirable.

The rate of the transdermal uptake of drugs is generally considered slow even with abrasion of the skin surface. The significant increase in rate provided by particular enhancers provides a rapid yet non-invasive method of drug delivery to achieve immediate results. Previous evidence would suggest that microprojections alone would be sufficient for rapid drug uptake since the stratum corneum has been breached. However, the inventors have found that microprojections alone result in an initial rapid uptake followed by a plateau effect resulting in a first order diffusion profile, potentially due to the rapid closure of the holes created by the microprojections. Incorporation of a penetration enhancer enables a further increase in uptake and maintenance of a first order diffusion profile.

In addition to providing improved percutaneous absorption efficiency, the method and drug delivery system of the invention may also provide lower irritancy and a reduced risk of infection than some other more invasive delivery systems such as intravenous injection, because the delivery system is non-invasive to the skin.

In another aspect the invention provides a method and apparatus which uses micro-projection apparatus comprising a cuff for encircling a body member; an array of projections extending inwardly from the cuff and means for applying a restrictive force for providing a predetermined depth of penetration of the micro-projections.

The transdermal composition used in accordance with this embodiment may and preferably will comprise a penetration enhancer in addition to the physiologically active agent. However there may be some circumstances in which the enhancer is not necessary for the required treatment or dose. Penetration enhancer is particularly preferred in this aspect and provides significant advantages.

In a preferred form of the invention, the cuff comprises an internal bladder that may be inflated to a predetermined pressure applying external pressure to a circumferential ring around the body member. Thereby, the micro-projections extending inwardly from the cuff may be inserted into the epidermis without reaching the free nerve endings for pain perception located in the basal layer of the viable epidermis or the vasculature system in the dermal layer. Previous studies have shown that holes which remained in the in-vitro skin after microneedles were removed were approximately 1 μm in size (Henry S et al, 1998, J Pharm Sci., 87(8); 922-925). Although reversibility was not reported, holes created by microneedles in vivo are likely to reseal, although the kinetics of resealing are presently unknown.

In one preferred form of the invention the micro-projections may penetrate from about 2 μm to about 800 μm into epidermal layer, more preferably from about 20 μm to about 500 μm into the epidermal layer.

In another preferred form of the invention the cuff may be inflated to exert a load force of between about 5 g/cm² and 500 g/cm² upon the array of micro-projections. In this manner, the pressure loading is increased at a consistent rate thereby avoiding any impact pressure loading and maintaining control over the depth of penetration of the micro-projections. More preferably the load force is between 20 g/cm² and 200 g/cm².

Whilst it is preferred that the micro-projection device and transdermal composition be administered simultaneously, the transdermal composition may be applied before or after the application of the micro-projection device, if desired.

In a further embodiment, the micro-projection device may further comprise a heating component, electromagnetic pump, iontophoresis, sonophoresis, electrophoresis, radio frequency, or a combination of any of the aforementioned mechanical forces.

A particular advantage of the transdermal composition of the present invention is the incorporation of one or more dermal penetration enhancers that may assist in the transport of the physiologically active agent across into the dermal layers, thereby further enhancing uptake into the systemic circulation via the vasculature or lymphatic system.

The dermal penetration enhancer may be selected from the classes of enhancers that are lipophilic non-volatile liquids whose vapour pressure is below 10 mm Hg at atmospheric pressure and normal skin temperature of 32 degrees Celsius. Preferably, the dermal penetration enhancer has a molecular weight within the range of 200 to 400 Daltons.

Examples of dermal penetration enhancers include: laurocapram (Azone®) and laurocapram derivatives, such as those 1-alkylazacycloheptan-2-ones specified in U.S. Pat. No. 5,196,410, and oleic acid and its ester derivatives, such as methyl, ethyl, propyl, isopropyl, butyl, vinyl and glycerylmonooleate, and sorbitan esters such as sorbitan monolaurate and sorbitan monooleate, and other fatty acid esters such as isopropyl laurate, isopropyl myristate, isopropyl palmitate, diisopropyl adipate, propylene glycol monolaurate and propylene glycol monooleate, and long chain alkyl esters of 2-pyrrolidone, particularly the 1-lauryl, 1-hexyl and 1-(2-ethylhexyl)esters of 2-pyrollidene and those dermal penetration enhancers given in U.S. Pat. No 6,299,900, particularly octyl salicylate, octyl dimethyl para-aminobenzoate and octyl para-methoxycinnamate (Padimate O), U.S. Pat. No. 5,082,866, particulary dodecyl (N,N-dimethylamino)acetate and dodecyl (N,N-dimethylamino)propionate and in U.S. Pat. No. 4,861,764, particularly 2-n-nonyl-1-3-dioxolane.

Preferred known dermal penetration enhancers are laurocapram and laurocapram derivatives, such as those 1-alkylazacycloheptan-2-ones specified in U.S. Pat. No. 5,196,410, and oleic acid and its ester derivatives, such as methyl, ethyl, propyl, isopropyl, butyl, vinyl and glycerylmonooleate, and those given in U.S. Pat. No. 5,082,866, particularly dodecyl (N,N-dimethylamino) acetate and dodecyl (N,N-dimethylamino)propionate and in U.S. Pat. No. 4,861,764, particularly 2-n-nonyl-1-3-dioxolane. Most preferred known dermal penetration enhancers are oleic acid and its ester derivatives, such as methyl, ethyl, propyl, isopropyl, butyl, vinyl and glycerylmonooleate, and those given in U.S. Pat. No. 6,299,900, particularly octyl salicylate, octyl dimethyl para-aminobenzoate and octyl para-methoxycinnamate (Padimate O), U.S. Pat. No. 5,082,866, particulary dodecyl (N,N-dimethylamino)acetate and dodecyl (N,N-dimethylamino)propionate and in U.S. Pat. No. 4,861,764, particularly 2-n-nonyl-1-3-dioxolane.

The present invention also provides a method for administering at least one systemic or locally acting physiologically active agent or prodrug thereof to an animal which comprises applying an effective amount of the physiologically active agent in the form of a drug delivery system according to the present invention. These physiologically active agents include, but are not limited to, macromolecules and hormones such as insulin, ACTH (corticotropin), parathyroid hormone, growth hormone (GH) and its analogues, GH antagonists, luteinizing hormone releasing hormone, follicle stimulating hormone, G-CSF, heparin, monoclonal antibodies, DNA polymers, genes and oligonucleotides, alpha-1 anti trypsin, anti-angiogenesis agents, anti-sense agents, butorphanol, calcitonin and its analogues, ceredase, COX-II inhibitors, dermatological agents, dihydroergotamine, dopamine agonists and antagonists, opioid peptides, analgesics including narcotic analgesics such as fentanyl, oligosaccharides, prostaglandins, sildenafil, thrombolytics, tissue plasminogen activators, RNF, vaccines, anti-tuberculosis agents, anti-addiction agents, anti-allergy agents, antiemetics and antinauseants such as granisetron and ondansetron, anti-obesity agents, anti-osteoporotics, anti-infectives, anaesthetics, anorexics, antiarthritics, antiasthmatic agents such as terbutaline, anticonvulsants, anti-depressants, anti-diabetic agents, antihistamines, anti-inflammatory agents including non-steroidal anti-inflammatory agents, anti-migraine agents, antineoplastics, antiparkinsonians, antipruritics including corticosteroids, antipsychotics, antipyretics, anticholinergics, benzodiazepine antagonists, vasodilators, antivirals.

Physiologically active agents applied to the skin in the absence of both micro-projections and dermal penetration enhancer result in a zero order release profile wherein the initial burst of physiologically active agent across the skin is limited. Physiologically active agents applied to the skin with the aid of micro-projections but in the absence of a dermal penetration enhancer will result in a rapid first order release profile that will plateau relatively quickly, thus reduce the therapeutic effect. In contrast, the combination of micro-projections, physiologically active agent and dermal penetration enhancer of the present invention is such that a rapid first order release rate profile can be achieved and maintained.

The release rate profile of the physiologically active agent from the composition into the systemic circulation preferably reaches maximum rate of flux within 6 hours, more preferably within 1 hour.

The amount of physiologically active agent administered will depend on a number of factors and will vary from subject to subject and depend on the particular physiologically active agent administered, the severity of the symptoms, the subject's age, weight and general condition, and the judgment of the prescribing physician. The minimum amount of physiologically active agent is determined by the requirement that sufficient quantities of the drug must be present in the composition to maintain the desired rate of release over the given period of application. The maximum amount for safety purposes is determined by the requirement that the quantity of drug present cannot exceed a rage of release that reaches toxic levels. Generally, the maximum concentration is determined by the amount of agent that can be received without producing adverse histological effects such as irritation. Of course it will be appreciated by those skilled in the art that the desired dose of a specific drug will depend on the nature of the drug as well as on other factors; the minimum effective dose of each physiologically active agent is of course preferred.

The present invention also provides for a transdermal drug delivery system which comprises:
a cuff for encircling a body member;
an array of micro-projections extending inwardly from the cuff;
a means for applying a predetermined constricting force to the cuff when placed about the body member thereby causing the array of micro-projections to penetrate the stratum corneum; and

a transdermal composition comprising:
a physiologically active agent;
at least one dermal penetration enhancer; and
a volatile liquid vehicle;
wherein the penetration of the stratum corneum facilitate transdermal penetration of the transdermal composition.

The device may be used prior to, during or after application of the transdermal composition.

The present invention also provides a transdermal drug delivery system which comprises a micro-projection apparatus for producing microholes in an area of skin and a transdermal composition comprising at least one physiologically active agent or prodrug thereof, preferably at least one dermal penetration enhancer and at least one volatile liquid; characterised in that the dermal penetration enhancer is a safe skin-tolerant ester sunscreen.

The transdermal composition preferably comprises:
(i) an effective amount of at least one physiologically active agent or prodrug thereof;
(ii) at least one non-volatile dermal penetration enhancer; and
(iii) at least one volatile liquid.

The dermal penetration enhancer is adapted to transport the physiologically active agent across a dermal surface or mucosal membrane of an animal, including a human, when the volatile liquid evaporates, to form a reservoir or depot of a mixture comprising the penetration enhancer and the physiologically active agent or prodrug within said surface or membrane; and

The dermal penetration enhancer is of low toxicity to, and is tolerated by, the dermal surface or mucosal membrane of the animal.

The present invention also provides a method for administering at least one systemic physiologically active agent or prodrug thereof to a animal which comprises topically applying an effective amount of the physiologically active agent to an area of skin to produce microholes.

Preferably the volatile liquid vehicle has a vapour pressure above 35 mm Hg at atmospheric pressure and normal skin temperature of 32 degrees Celsius. In a particularly preferred form of the invention the liquid is ethanol, ethyl acetate or isopropanol, or mixture thereof in the range of about 40 to 99%. An aerosol propellant, such as dimethyl ether, may constitute a volatile liquid for the purpose of the present invention.

In drug delivery systems according to the present invention a pharmaceutical compounding agent, co-solvent, surfactant, emulsifier, antioxidant, preservative, stabiliser, diluent or a mixture of two or more of said components may be incorporated in these systems as is appropriate to the particular dosage form. The amount and type of components used should be compatible with the dermal penetration enhancers of this invention as well as with the active ingredient. A co-solvent or other standard adjuvant, such as a surfactant, may be required to maintain the agent in solution or suspension at the desired concentration.

Preferably the animal is a human but the invention also extends to the treatment of non-human animals.

Preferably the transdermal composition is not supersaturated with respect to the physiologically active agent or prodrug. As the volatile liquid of the non-occlusive drug delivery system evaporates, the resulting non-volatile composition is rapidly driven into the dermal surface or mucosal membrane. It is possible that as the volatile liquid evaporates, the non-volatile dermal penetration enhancer becomes supersaturated with respect to the active agent. However, it is preferred that any supersaturation does not occur before transport of the resulting non-volatile composition across the epidermal surface has occurred.

It is most desirable that, after application of the non-occlusive, percutaneous or transdermal drug delivery system, the volatile component of the delivery system evaporates and the area of skin to which the drug delivery system was applied becomes touch-dry. Preferably said area of skin becomes touch-dry within 10 minutes, more preferably within 3 minutes, most preferably within 1 minute.

Preferred volatile liquids of the present invention include safe skin-tolerant solvents such as ethanol and isopropanol. An aerosol propellant, such as dimethyl ether, or an HFC such as R134a may constitute a volatile liquid for the purpose of the present invention.

The transdermal composition may contain additives such as pharmaceutical compounding agent, co-solvent, surfactant, emulsifier, antioxidant, preservative, stabiliser, diluent or a mixture of two or more of said components may be incorporated in these systems as is appropriate to the particular route of administration and dosage form. The amount and type of components used should be compatible with the dermal penetration enhancers of this invention as well as with the active ingredient. A co-solvent or other standard adjuvant, such as a surfactant, may be required to maintain the agent in solution or suspension at the desired concentration.

The pharmaceutical compounding agents can include paraffin oils, esters such as isopropyl myristate, ethanol, silicone oils and vegetable oils. These are preferably used in the range 1 to 50%. Surfactants such as ethoxylated fatty alcohols, glycerol mono stearate, phosphate esters, and other commonly used emulsifiers and surfactants preferably in the range of 0.1 to 10% may be used, as may be preservatives such as hydroxybenzoate esters for preservation of the compound preferably in amounts of 0.01% to 0.5%. Typical co-solvents and adjuvants may be ethyl alcohol, isopropyl alcohol, acetone, dimethyl ether and glycol ethers such as diethylene glycol mono ethyl ether. These may be used in amounts of 1 to 50%.

In drug delivery systems according to the second aspect of the present invention, whilst a pharmaceutical compounding agent, co-solvent, surfactant, emulsifier, antioxidant, preservative, stabiliser, diluent or a mixture of two or more of said components may be incorporated, it is particularly preferred that these be selected so as to be compatible with the ability of the system becoming touch-dry after application.

Because of the efficiency of the method of the invention the dosage of the physiologically active agent may often be less than that conventionally used. It is proposed that, a dosage near the lower end of the useful range of the particular agent may be employed initially and increased as indicated from the observed response if necessary.

The concentration of physiologically active agent used in the drug delivery system will depend on its properties and may be equivalent to that normally utilised for the particular agent in conventional formulations. Both the amount of physiologically active agent and the amount of penetration enhancer will be influenced by the type of effect desired. For example, if a more localised effect is required in treating a superficial infection with an antibacterial agent, lower amounts of physiologically active agents and lower concentrations of enhancer may be appropriate. Where deeper penetration is desired, as in the case of local anaesthesia, a higher concentration of enhancer may be appropriate.

Where it is desired to achieve systemic concentration of an agent, proportionately higher concentrations of the enhancer of the invention may be required in the transdermal drug delivery system of the present invention, and the amount of active substance included in the composition should be sufficient to provide the blood level desired.

The concentration of absorption/penetration enhancer may be in the range from 10-10,000 weight percent of absorption/penetration enhancer based upon the weight of active ingredient. The ratio of penetration enhancer to active ingredient may vary considerably and will be governed as much as anything, by the pharmacological results that are required to be achieved. In principle, it is desirable that as little absorption enhancer as possible is used. On the other hand, for some actives, it may well be that the upper range of 10,000% by weight will be required. It is preferred that the penetration enhancer and active are in approximately equal proportions.

Surprisingly, it has been found that a large range of systemic drugs can be rapidly delivered to a subject in need thereof by the methods of the present invention.

The drug delivery system of the present invention may be applied to the skin by means of an aerosol, spray, pump-pack, brush, swab, or other applicator. Preferably, the applicator provides either a fixed or variable metered dose application such as a metered dose aerosol, a stored-energy metered dose pump or a manual metered dose pump.

The drug delivery system may be propelled by either pump pack or more preferably by the use of propellants such as hydrocarbons, hydro fluorocarbons, nitrogen, nitrous oxide, carbon dioxide or ethers, preferably dimethyl ether. The non-occlusive, drug delivery system is preferably in a single phase system as this allows less complicated manufacture and ease of dose uniformity. It may also be necessary to apply a number of dosages on untreated skin to obtain the desired result.

### BRIEF DESCRIPTION OF THE FIGURES

In the accompanying figures:

FIG. 1 Shows the cumulative amount of fentanyl penetrating across human epidermis (μg/cm²) versus time (hours) for the topical solution composition A after varying pressure loading. Error bars represent Standard Error of the Mean (SEM).

FIG. 2 Shows the cumulative amount of fentanyl penetrating across human epidermis (μg/cm²) versus time (hours) for topical solution compositions A and B with or without microneedle penetration and the dermal penetration enhancer, octyl salicylate. Error bars represent Standard Error of the Mean (SEM).

FIG. 3 Shows the cumulative amount of fentanyl penetrating across human epidermis (μg/cm²) versus time (hours) for topical solution compositions A with microneedle penetration, the dermal penetration enhancer, octyl salicylate and increasing temperature. Error bars represent Standard Error of the Mean (SEM).

FIG. 4 Shows a plan view of the underside of a microprojection apparatus in accordance with a preferred embodiment of the, invention.

FIG. 5 Shows a cross-section of the microprojection apparatus of FIG. 4 along the line V-V.

FIG. 6 Shows a front elevation view partially illustrating the microprojection apparatus of FIG. 4 as applied to the upper arm of the human body

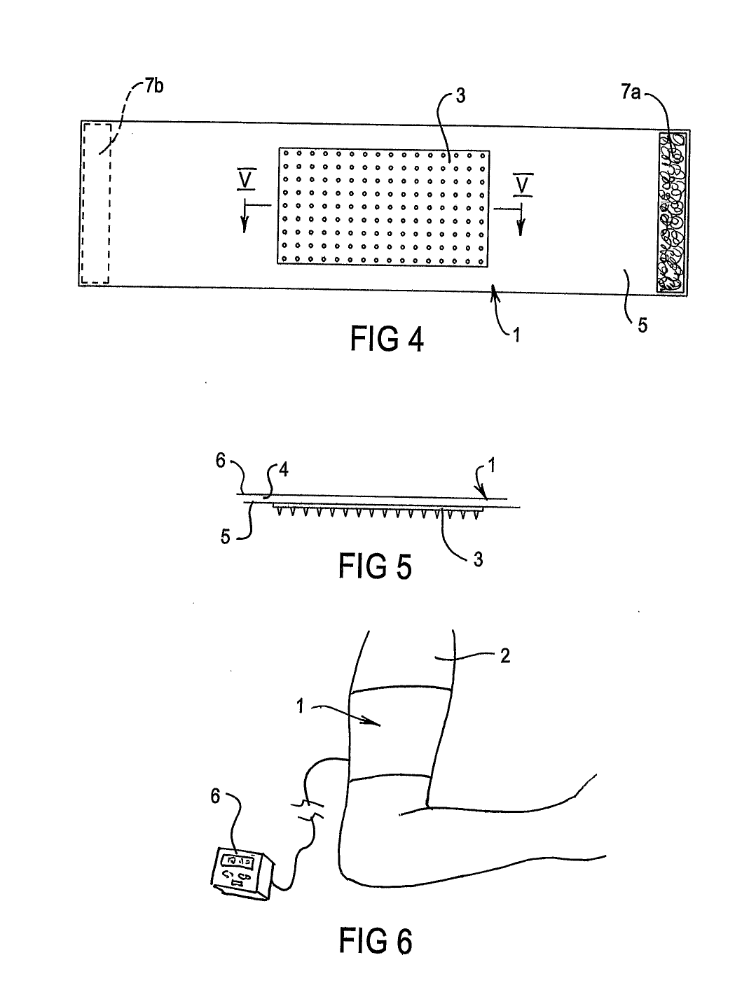

In FIGS. 4 to 6 a cuff (1) is provided for encircling a human upper arm (2). The cuff (1) is provided with an array of microprojections (3) (preferably of between 50 microns and 2 mm in length) supported thereon so as to project inwardly from the cuff (1) when used to encircle the upper arm (2). The cuff may be provided with cooperating portions fastener (7a, 7b) such as buttons and holes or complementary hook and eye fastener portions or the like for locking it in position about the upper arm. The cuff (2) includes a reservoir (4) formed by opposing layers (5,6) adapted to receive a fluid therebetween such as a gas or liquid to inflate the cuff (1) and constrict the inner layer (5) of the cuff (1) to force the array of microprojections against the upper arm. The apparatus is preferably provided with inflation means (6) such as a manual or automated air pump for inflating the cuff (1) to provide the predetermined constricting force. The inflation means is in fluid communication with the internal reservoir (4) of the cuff (1) to allow introduction of fluid and/or pressure.

In operation the transdermal compositions may be applied to the skin of the upper arm (2) in the area to be contacted by the microprojections (3). Alternatively the transdermal composition may be applied to the array of microprojections (3) or applied to the microholes created in an area of skin of the upper arm (2) following use of the cuff.

In describing the present invention, the following terminology will be used in accordance with the definitions set out below.

The term “stratum corneum” is used herein in its broadest sense to refer to the outer layer of the skin, which is comprised of (approximately 15) layers of terminally differentiated keratinocytes made primarily of the proteinaceous material keratin arranged in a ‘brick and mortar’ fashion with the mortar being comprised of a lipid matrix made primarily from cholesterol, ceramides and long chain fatty acids. The stratum corneum creates the rate-limiting barrier for diffusion of the active agent across the skin.

The term “dermal penetration enhancer” is used herein in its broadest sense to refer to an agent which improves the rate of percutaneous transport of active agents across the skin for use and delivery of active agents to organisms such as animals, whether it be for local application or systemic delivery.

The term “physiologically active agent” is used herein to refer to a broad class of useful chemical and therapeutic agents.

The term “physiologically active” in describing the agents contemplated herein is used in a broad sense to comprehend not only agents having a direct pharmacological effect on the host, but also those having an indirect or observable effect which is useful in the medical arts.

It is believed that the rate of initial uptake of the physiologically active agent into the systemic circulation is rapidly enhanced by the application of the micro-projection device. The holes created in the stratum corneum close over a short period of time, and thus uptake can be further enhanced by the incorporation of one or more dermal penetration enhancers in the composition.

The invention will now be described with reference to the following examples. It is to be understood that the examples are provided by way of illustration of the invention and that they are in no way limiting to the scope of the invention.

### EXAMPLES

In the examples the following transdermal compositions were studied to examine the effective use of micro-projections.

Study Treatments

| | | |
|---|---|---|
| A | Fentanyl | 5% |
| | Octyl salicylate | 5% |
| | Alcohol USP (95%) | to volume |

| | | |
|---|---|---|
| B | Fentanyl | 5% |
| | Alcohol USP (95%) | to volume |

### Example 1

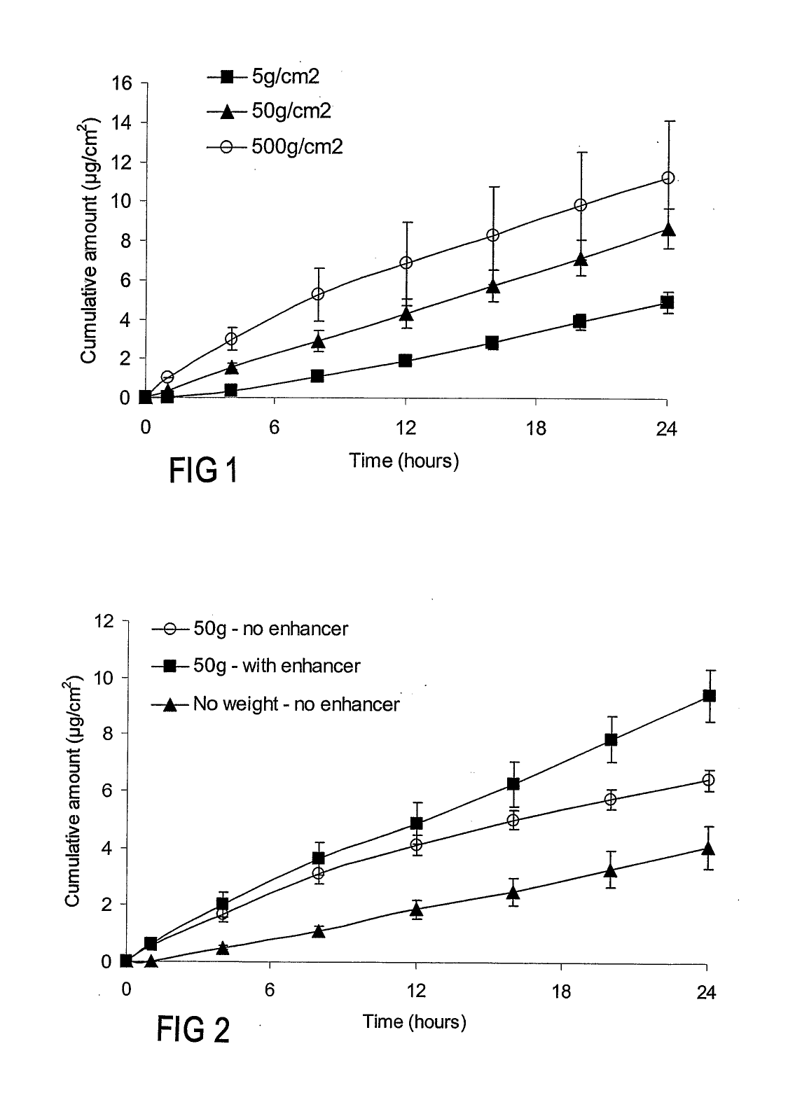

As shown in FIG. 1, an increase in pressure loading on the micro-projections caused an increase in the transdermal delivery of fentanyl across the skin.

The diffusion experiments were performed using human epidermis as the model membrane. The epidermis was backed onto filter paper for additional support. These experiments were performed over 24 h with stainless steel, flow-through diffusion cells based on those previously described, (Cooper, E. R. J. Pharm. Sci. 1984, 73, 1153-1156.) except that the cell was modified to increase the diffusional area to 1.0 cm². Three different pressure loads with micro-projections were applied to the diffusion cells, 5 g, 50 g or 500 g, for a period of 1 minute. A finite dose of 5 μl/cm² of formulation A was applied to the diffusion cell and left uncovered for the diffusion of the experiment. A piece of stainless steel wire mesh was placed directly below the skin in the receptor chamber of the diffusion cell to maintain a turbulent flow of receptor solution below the skin. The diffusion cells were maintained at a flow rate of approximately 1.0 mL/cm²/h by a microcassette peristaltic pump (Watson Marlow 505S, UK). The cells were kept at 32±0.5° C. by a heater bar and the samples are collected into appropriately sized plastic vials on an automated fraction collector (Isco Retriever II, Lincoln, NE) at specified intervals. The receptor solution (20% EtOH with 0.002% sodium azide) maintained sink conditions beneath the skin.

Samples were analysed by RP-HPLC using the following conditions; Column-Waters Symmetry C₁₈ column (3.9×150 mm) with a 5 μm support size; Mobile phase—80% AcN in aqueous 0.009% PCA with 9 mM 1-HAS, 20% AcN; Flow rate—1.0 mL/min; Absorbance—210 nm; and Injection volume—50 μL.

### Example 2

As shown in FIG. 2 the addition of a dermal penetration enhancer, octyl salicylate, resulted in a further increase in the transdermal delivery of fentanyl across the skin (p<0.01) following application of a 50 g pressure loading.

The diffusion experiments were performed according to example 1, except that a 50 g pressure load with micro-projections was applied to the cell and a finite dose of 5 μl/cm² of either formulation A or formulation B was applied to the diffusion cell.

### Example 3

Diffusion experiments were performed according to example 1, except that a 50 g pressure load with micro-projections were applied to the cell, a finite dose of 5 μl/cm² of formulation A was applied to the diffusion cell and the cell temperature was set at either 32° C., 38° C. or 45° C.

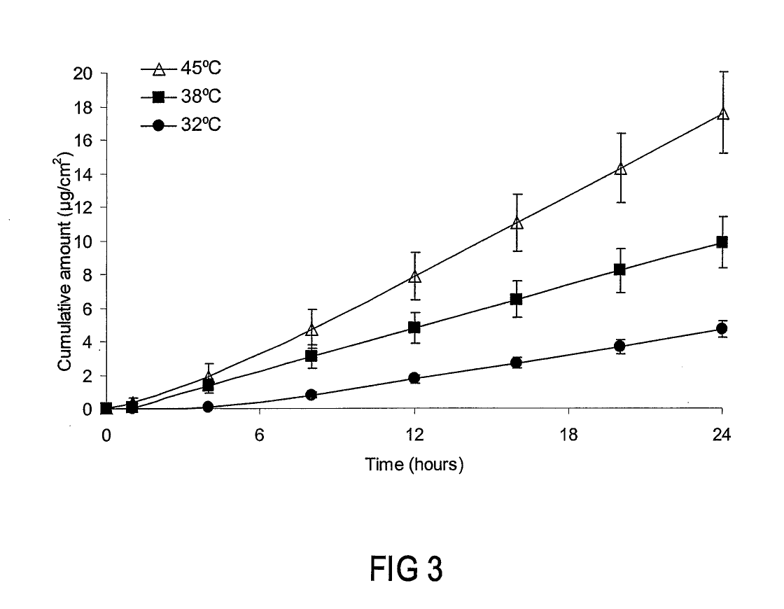

As shown in FIG. 3, the increasing temperature resulted in a further increase in the transdermal delivery of fentanyl across the skin (p<0.01) following application of microprojections under a 50 g pressure loading and formulation A.

The results show that microprojections provide a rapid first order absorption profile with reduced long term effect. This is important in achieving pain relief and in other treatments where rapid response is desirable.

1. A method for transdermal delivery of a topically applied physiologically active agent comprising:
providing a micro-projection apparatus comprising an array of micro-projections extending from a substrate;
applying the array of micro-projections to an area of skin to form an array of microscopic holes therein; and
contacting the area of skin with the transdermal composition comprising a physiologically active agent and at least one penetration enhancer
wherein the formation of the microscopic holes and penetration enhancer facilitate transdermal delivery of the physiologically active agent.

2. A method for transdermal delivery of a physiologically active agent topically applied to an area of skin of a body member, which method comprises:
providing a micro-projection apparatus comprising:
a cuff for encircling a body member;
an array of micro-projections extending inwardly from at least a portion of the cuff for providing microscopic holes in the area of skin; and
means for applying a constricting force to the cuff when placed about the body member; and
providing a transdermal composition comprising:
a physiologically active agent; and
at least one dermal penetration enhancer;
topically applying the transdermal composition to an area of skin of the body member;
applying the cuff about the body member and activating the means for applying the constricting force thereby causing the array of micro-projections to penetrate the stratum corneum of the area of skin wherein the micro-projection apparatus and penetration enhancer facilitate transdermal penetration of the transdermal composition.

3. A method for transdermal delivery according to claim 2 wherein the cuff comprises an internal bladder that may be inflated to a predetermined pressure to thereby urge the array of micro-projections against the body member.

4. A method for transdermal delivery for transdermal delivery according to claim 1 wherein the micro-protrusions are caused to penetrate to a depth of from about 2 μm to about 800 μm into epidermal layer.

5. A method for transdermal delivery according to claim 4 wherein the projections are caused to penetrate from about 20 μm to about 500 μm into the epidermal layer.

6. A method for transdermal delivery according to claim 3 wherein the cuff is inflatable and is inflated about the body member to exert a load force of between about 5 g/cm² and 500 g/cm² upon the array of micro-projections.

7. A method for transdermal delivery according to claim 6 wherein the load force is between 20 g/cm² and 200 g/cm².

8. A method for transdermal delivery according to claim 1 wherein the transdermal composition is applied to the area of skin prior to applying the array of micro-projections.

9. A method for transdermal delivery according to claim 1 wherein the transdermal composition further comprises a volatile organic solvent.

10. A method for transdermal delivery according to claim 9 wherein the area of skin is touch dry within 3 minutes of application of the transdermal composition.

11. A method for transdermal delivery according to claim 3 wherein the cuff comprises a heating component, electromagnetic pump, iontophoresis, sonophoresis, electrophoresis, radio frequency, or a combination of two or more thereof.

12. A method for transdermal delivery according to claim 1 wherein heat is applied to the area of skin in the presence of the transdermal composition.

13. A method for transdermal delivery according to claim 1 wherein the transdermal composition comprises at least one dermal penetration enhancer selected from the group consisting of laurocapram and laurocapram derivatives, fatty acid esters, sunscreen esters, long chain alkyl(N,N-disubstituted amino)carboxylates, 1,3-dioxacyclopentanes, and 1,3-dioxacyclohexanes.

14. A method for transdermal delivery according to claim 1 3 wherein the at least one transdermal penetration enhancer is selected from the group consisting of: laurocapram and laurocapram derivatives selected from the group consisting of 1-alkylazacycloheptan-2-ones and oleic acid and its ester derivatives, selected from methyl, ethyl, propyl, isopropyl, butyl and vinyl vinyl ester derivatives; glycerylmonooleate; long-chain alkyl(N,N-dialkylaminocarboxylates selected from the group consisting of dodecyl (N,N-dimethylamino)acetate and dodecyl (N,N-dimethylamino)propionate; sunscreen esters selected from the group consisting of octyl salicylate, octyl dimethyl para-aminobenzoate and octyl para-methoxycinnamate (Padimate O); and 2-n-nonyl-1-3, dioxolane.

15. A method for transdermal delivery according to claim 14 wherein the penetration enhancer comprises a sunscreen ester.

16. A method for transdermal delivery according to claim 1 wherein the physiologically active agent comprises at least one agent selected from the group consisting of hormones, GH antagonists, luteinizing hormone releasing hormone, follicle stimulating hormone, G-CSF, heparin, monoclonal antibodies, DNA polymers, genes and oligonucleotides, alpha-1 anti trypsin, anti-angiogenesis agents, anti-sense agents, butorphanol, calcitonin and its analogues, ceredase, COX-II inhibitors, dermatological agents, dihydroergotamine, dopamine agonists and antagonists, opioid peptides, analgesics including narcotic analgesics such as fentanyl, oligosaccharides, prostaglahdins, sildenafil, thrombolytics, tissue plasminogen activators, RNF, vaccines, anti-tuberculosis agents, anti-addiction agents, anti-allergy agents, antiemetics and antinauseants, anti-obesity agents, anti-osteoporotics, anti-infectives, anaesthetics, anorexics, antiarthritics, antiasthmatics, anticonvulsants, anti-depressants, anti-diabetic agents, antihistamines, anti-inflammatory agents including non-steroidal anti-inflammatory agents, anti-migraine agents, antineoplastics, antiparkinsonians, antipruritics including corticosteroids, antipsychotics, antipyretics, anticholinergics, benzodiazepine antagonists, vasodilators and antivirals.

17. A method for transdermal delivery according to claim 1 wherein the transdermal composition is applied to the area of skin as a spray.

18. A system for transdermal delivery of a topically applied physiologically active agent which comprises:
a micro-projection apparatus comprising:
a cuff for encircling a body member;
an array of micro-projections extending inwardly from the cuff;
a means for applying a predetermined constricting force to the cuff when placed about the body member thereby causing the array of micro-projections to penetrate the stratum corneum; and
a transdermal composition comprising:
a physiologically active agent; and
at least one dermal penetration enhancer; and
wherein the micro-projection apparatus facilitates transdermal penetration of the transdermal composition.

19. A system for transdermal delivery according to claim 17 wherein the cuff comprises an internal bladder that may be inflated to a predetermined pressure applying external pressure to a circumferential ring around the body member.
